(19) United States
(12) Patent Application Publication
(10) Pub. No.: US 2004/0163139 A1
(43) Pub. Date: Aug. 19, 2004

(54) Conus gamma-carboxylase

(75) Inventors: James E. Garrett, Salt Lake City, UT (US); Pradip K. Bandyopadhyay, Salt Lake City, UT (US)

Correspondence Address: ROTHWELL, FIGG, ERNST & MANBECK, P.C., 1425 K STREET, N.W., SUITE 800, WASHINGTON, DC, 20005, US

(73) Assignees: Cognetix, Inc., Salt Lake City, UT (US); University of Utah Research Foundation, Salt Lake City, UT (US)

(21) Appl. No.: 10/788,266

(22) Filed: Mar. 1, 2004

Related U.S. Application Data
(63) Continuation of application No. 10/213,439, filed on Aug. 7, 2002, now abandoned.
(60) Provisional application No. 60/310,496, filed on Aug. 8, 2001.

(51) Int. Cl. A01K 67/033; C07H 21/04; C12N 9/48

(52) U.S. Cl. 800/8; 435/69.1; 435/212; 435/320.1; 435/325; 536/23.2

(57) ABSTRACT

The present invention is relates to a γ-carboxylase from Conus snails, a nucleic acid sequence encoding the Conus γ-carboxylase and to a method for using the nucleic acid or protein sequences for preparing γ-carboxylated proteins.

### CROSS-REFERENCE TO RELATED APPLICATION

The present application is a continuation of U.S. patent application Ser. No. 10/213,439 filed 7 Aug. 2002. The present application is related to and claims priority under 35 U.S.C. §119(e) to U.S. provisional patent application Ser. No. 60/310,496 filed 8 Aug. 2001, incorporated herein by reference.

This invention was made with Government support under Grant No. PO1 GM48677 awarded by the National Institute of General Medical Sciences, National Institutes of Health, Bethesda, Md. The United States Government has certain rights in the invention.

### BACKGROUND OF THE INVENTION

The present invention relates to a γ-carboxylase from Conus snails, a nucleic acid sequence encoding the Conus γ-carboxylase and to a method for using the nucleic acid or protein sequences for preparing γ-carboxylated proteins.

The publications and other materials used herein to illuminate the background of the invention, and in particular, cases to provide additional details respecting the practice, are incorporated by reference, and for convenience are referenced in the following text by number and are listed numerically in the appended Bibliography.

The vitamin K-dependent γ-carboxylation of glutamate residues was originally discovered as a novel post-translational modification in the blood coagulation cascade (Stenflo et al., 1974); some of the key clotting factors such as prothrombin must be γ-carboxylated in order for proper blood clotting to occur. Somewhat later, this post-translational modification was also found in certain bone proteins (Price and Williamson, 1985). In mammalian blood coagulation and bone Gla proteins, γ-carboxylation of glutamate residues is carried out by a vitamin K-dependent carboxylase. A conserved motif (Price et al., 1987) γ-carboxylation recognition sequence in the propeptide sequence binds the γ-carboxylase and is required for a polypeptide substrate to be a high affinity target for the γ-carboxylase.

This modification was restricted to these rather specialized mammalian systems until a very unusual peptide, conantokin-G, was described from the venom of the predatory marine snail, Conus geographus (McIntosh et al., 1984). Conantokin-G is a 17-amino acid peptide that inhibits the N-methyl- D-aspartate receptor (Olivera et al., 1990). Unlike most Conus peptides, which are multiply disulfide-bonded, conantokin-G has no disulfide cross-links but has five residues of γ-carboxyglutamate residues; this remains the highest density of γ-carboxyglutamate found in any functional gene product characterized to date. Most of the biologically active components of the Conus venom are multiply disulfide bonded peptides (the conotoxins). These have been shown to be initially translated as prepropeptide precursors, which are then post-translationally processed to yield the mature disulfide-crosslinked conotoxin. Conantokin-G differs strikingly from most conotoxins not only in having γ-carboxyglutamate residues, but also because it has no disulfide crosslinks. U.S. Pat. No. 6,197,535 describes the analysis of the conantokin-G precursor and sequence recognition by a γ-carboxylase for the maturation of the functional conantokin-G peptide. It was found a γ-carboxylation recognition sequence is included in the −1 to −20 region of the conantokin-G prepropeptide. This sequence appears to increase the affinity of the Conus carboxylase by approximately two orders of magnitude.

The presence of γ-carboxyglutamate in a non-mammalian system was initially controversial because vitamin K-dependent carboxylation of glutamate residues had primarily been thought to be a highly specialized mammalian innovation. However, conantokin-G is only one member of a family of peptides; a variety of other conantokins have been found including conantokin-T and conantokin-R from two other fish-hunting cone snails (Haack et al., 1990; White et al., 1997). All three peptides have a high content of γ-carboxyglutamate (4-5 residues). γ-Glutamyl carboxylase has been purified from mammalian sources (Wu et al., 1991a; Berkner et al., 1992), has been expressed both in mammalian and insect cell lines (Wu et al., 1991b; Roth et al., 1993) and has been purified from Conus (U.S. Pat. No. 6,197,535). Recently it was shown that, as is the case in the mammalian system, the carboxylation reaction in Conus venom ducts absolutely requires vitamin K, and the net carboxylation increases greatly in the presence of high concentrations of ammonium sulfate. In these respects, the mammalian and the Conus γ-carboxylation venom systems are very similar (Stanley et al., 1997).

Knobloch and Suttie (1987) and Cheung et al. (1989) found that the propeptide sequences of Factors IX and X at micromolar concentrations stimulated the carboxylation of oligopeptide substrates, suggesting a probable positive allosteric effector role. In addition, the propeptide at micromolar concentrations acted as a competitive inhibitor of carboxylation of a substrate whose sequences were based on residues −18 to +10 of prothrombin (Ulrich et al., 1988). Similarly, the Conus propeptide (−20 to −1) inhibits the carboxylation of propeptide-containing substrates, (e.g., −10.Pro-E.Con-G and −20.Pro-E.Con-G) (U.S. Pat. No. 6,197,535).

The orientation in which a Glu presents itself to the active site of the carboxylase may determine whether it will be carboxylated. In the case of Con-G not all the Glu residues are γ-carboxylated (e.g., Glu² is not carboxylated, whereas Glu³ and Glu⁴ are carboxylated). The solution structures of Con-G and Con-T as determined by CD and NMR spectroscopy (Skjaebaek et al., 1997; Warder et al., 1997) are a mixture of α and 3₁₀ helices. Rigby et al. (1997) also determined the structure of the metal-free conformer of conantokin-G by NMR spectroscopy. In all of these structures, the Gla residues are on the same side of the conantokin structure; this would allow a membrane-bound enzyme to carry out efficient carboxylation of Glu residues oriented in the same direction with optimum stereochemistry.

There is a need in the art to identify the nucleic acid sequence encoding Conus γ-carboxylase, to identify the sequence of Conus γ-carboxylase, and to use the nucleic acids or proteins in the production of γ-carboxylated proteins.

### SUMMARY OF THE INVENTION

The present invention is relates to a γ-carboxylase from Conus snails, a nucleic acid sequence encoding the Conus γ-carboxylase and to a method for using the nucleic acid or protein sequences for preparing γ-carboxylated proteins.

Thus, one aspect of the invention is directed to the amino acid sequence of C. textile γ-carboxylase. The amino acid sequence of C. textile γ-carboxylase is set forth in SEQ ID NO:2 or SEQ ID NO:4.

A second aspect of the invention is directed to a nucleic acid encoding a C. textile γ-carboxylase. A preferred nucleotide sequence of the nucleic acid is set forth in SEQ ID NO:1 or SEQ ID NO:3.

A third aspect of the invention is directed to amino acid sequences and nucleic acid sequences of other Conus γ-carboxylases, as well as amino acid sequences and nucleic acid sequences having 95% identity with the disclosed sequences.

A fourth aspect of the invention is directed to vectors containing the γ-carboxylase encoding nucleic acid.

A fifth aspect of the invention is directed to host cells containing an expression cassette with the γ-carboxylase encoding nucleic acid.

A sixth aspect of the invention is directed to host cells containing an expression cassette with the γ-carboxylase encoding nucleic acid sequence and an expression cassette with a nucleic acid sequence encoding a protein which is γ-carboxylated. Such proteins include conantokins and other vitamin K-dependent proteins.

A seventh aspect of the invention is directed to the use of a γ-carboxylase for the preparation of γ-carboxylated proteins (the term used herein to refer to proteins which are γ-carboxylated), such as conantokins and other vitamin K-dependent proteins.

An eighth aspect of the invention is directed to the use of a γ-carboxylase nucleic acid for the preparation of γ-carboxylated proteins, such as conantokins and other vitamin K-dependent proteins.

### DETAILED DESCRIPTION OF THE INVENTION

The present invention is relates to a γ-carboxylase from Conus snails, a nucleic acid sequence encoding the Conus γ-carboxylase and to a method for using the nucleic acid or protein sequences for preparing γ-carboxylated proteins.

In one aspect, the present invention relates to the amino acid sequence of C. textile γ-carboxylase. The amino acid sequence of C. textile γ-carboxylase is set forth in SEQ ID NO:2 or SEQ ID NO:4. In a further embodiment, the present invention relates to a γ-carboxylase which has at least 95% identity with the amino acid sequence set forth in SEQ ID NO:2 or SEQ ID NO:4 and which has γ-carboxylation activity. The γ-carboxylation activity can be assayed as described herein to identify those proteins having the proper biological activity.

In a second aspect, the present invention relates to a nucleic acid encoding a C. textile γ-carboxylase. A preferred nucleic acid sequence is set forth in SEQ ID NO:1 or SEQ ID NO:3. In a further embodiment, the present invention relates to a γ-carboxylase encoding nucleic acid which has at least 95% identity with the nucleotide sequence set forth in SEQ ID NO:1 or SEQ ID NO:3. The encoded γ-carboxylase has γ-carboxylation activity which can be assayed as described herein to identify those nucleic acids which encode proteins having the proper biological activity.

In a third aspect, the present invention relates to vectors containing the nucleic acid encoding a γ-carboxylase of the present invention. In one embodiment, the vector is an expression vector.

In a fourth aspect, the present invention relates to host cells containing an expression cassette or expression vector with the γ-carboxylase encoding nucleic acid of the present invention. The host cells produce the γ-carboxylase when grown under suitable growth conditions.

In a fifth aspect, the present invention relates to host cells containing an expression cassette or expression vector with the γ-carboxylase encoding nucleic acid of the present invention and an expression cassette with a nucleic acid sequence encoding a protein which is γ-carboxylated. Such proteins include conantokins and other vitamin K-dependent proteins.

In a sixth aspect, the present invention relates to the use of a γ-carboxylase of the present invention for the preparation of γ-carboxylated proteins, such as conantokins and other vitamin K-dependent proteins.

In a seventh aspect, the present invention relates to the use of a γ-carboxylase encoding nucleic acid of the present invention for the preparation of γ-carboxylated proteins, such as conantokins and other vitamin K-dependent proteins.

A nucleic acid or fragment thereof has substantial identity with another if, when optimally aligned (with appropriate nucleotide insertions or deletions) with the other nucleic acid (or its complementary strand), there is nucleotide sequence identity in at least about 60% of the nucleotide bases, usually at least about 70%, more usually at least about 80%, preferably at least about 90%, and more preferably at least about 95-98% of the nucleotide bases. A protein or fragment thereof has substantial identity with another if, optimally aligned, there is an amino acid sequence identity of at least about 30% identity with an entire naturally-occurring protein or a portion thereof, usually at least about 70% identity, more usually at least about 80% identity, preferably at least about 90% identity, and more preferably at least about 95-98% identity.

Identity means the degree of sequence relatedness between two polypeptide or two polynucleotides sequences as determined by the identity of the match between two strings of such sequences, such as the full and complete sequence. Identity can be readily calculated. While there exist a number of methods to measure identity between two polynucleotide or polypeptide sequences, the term “identity” is well known to skilled artisans (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M Stockton Press, New York, 1991). Methods commonly employed to determine identity between two sequences include, but are not limited to those disclosed in Guide to Huge Computers, Martin J. Bishop, ed., Academic Press, San Diego, 1994, and Carillo, H., and Lipman, D., SIAM J Applied Math. 48: 1073 (1988). Preferred methods to determine identity are designed to give the largest match between the two sequences tested. Such methods are codified in computer programs. Preferred computer program methods to determine identity between two sequences include, but are not limited to, GCG (Genetics Computer Group, Madison Wis.) program package (Devereux, J., et al., Nucleic Acids Research 12(1). 387 (1984)), BLASTP, BLASTN, FASTA (Altschul et al. (1990); Altschul et al. (1997)). The well-known Smith Waterman algorithm may also be used to determine identity.

As an illustration, by a polynucleotide having a nucleotide sequence having at least, for example, 95% “identity” to a reference nucleotide sequence of is intended that the nucleotide sequence of the polynucleotide is identical to the reference sequence except that the polynucleotide sequence may include up to five point mutations per each 100 nucleotides of the reference nucleotide sequence. In other words, to obtain a polynucleotide having a nucleotide sequence at least 95% identical to a reference nucleotide sequence, up to 5% of the nucleotides in the reference sequence may be deleted or substituted with another nucleotide, or a number of nucleotides up to 5% of the total nucleotides in the reference sequence may be inserted into the reference sequence. These mutations of the reference sequence may occur at the 5 or 3 terminal positions of the reference nucleotide sequence or anywhere between those terminal positions, interspersed either individually among nucleotides in the reference sequence or in one or more contiguous groups within the reference sequence.

Alternatively, substantial homology or (similarity) exists when a nucleic acid or fragment thereof will hybridize to another nucleic acid (or a complementary strand thereof) under selective hybridization conditions, to a strand, or to its complement. Selectivity of hybridization exists when hybridization which is substantially more selective than total lack of specificity occurs. Typically, selective hybridization will occur when there is at least about 55% homology over a stretch of at least about 14 nucleotides, preferably at least about 65%, more preferably at least about 75%, and most preferably at least about 90%. The length of homology comparison, as described, maybe over longer stretches, and in certain embodiments will often be over a stretch of at least about nine nucleotides, usually at least about 20 nucleotides, more usually at least about 24 nucleotides, typically at least about 28 nucleotides, more typically at least about 32 nucleotides, and preferably at least about 36 or more nucleotides.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. However, the combination of parameters is much more important than the measure of any single parameter. The stringency conditions are dependent on the length of the nucleic acid and the base composition of the nucleic acid, and can be determined by techniques well known in the art. See, e.g., Asubel, 1992; Wetmur and Davidson, 1968.

Thus, as herein used, the term “stringent conditions” means hybridization will occur only if there is at least 95% and preferably at least 97% identity between the sequences. Such hybridization techniques are well known to those of skill in the art. Stringent hybridization conditions are as defined above or, alternatively, conditions under overnight incubation at 42° C. in a solution comprising: 50% formamide, 5×SSC (150 mM NaCl, 15 mM trisodium citrate), 50 mM sodium phosphate (pH7.6), 5×Denhardt's solution, 10% dextran sulfate, and 20 μg/ml denatured, sheared salmon sperm DNA, followed by washing the filters in 0.1×SSC at about 65° C.

The terms “isolated”, “substantially pure”, and “substantially homogeneous” are used interchangeably to describe a protein or polypeptide which has been separated from components which accompany it in its natural state. A monomeric protein is substantially pure when at least about 60 to 75% of a sample exhibits a single polypeptide sequence. A substantially pure protein will typically comprise about 60 to 90% W/W of a protein sample, more usually about 95%, and preferably will be over about 99% pure. Protein purity or homogeneity may be indicated by a number of means well known in the art, such as polyacrylamide gel electrophoresis of a protein sample, followed by visualizing a single polypeptide band upon staining the gel. For certain purposes, higher resolution may be provided by using HPLC or other means well known in the art which are utilized for purification.

Large amounts of the nucleic acids of the present invention may be produced by (a) replication in a suitable host or transgenic animal or (b) chemical synthesis using techniques well known in the art. Nucleic acids made by either of these techniques are also referred to as synthetic nucleic acids herein. Constructs prepared for introduction into a prokaryotic or eukaryotic host may comprise a replication system recognized by the host, including the intended polynucleotide fragment encoding the desired polypeptide, and will preferably also include transcription and translational initiation regulatory sequences operably linked to the polypeptide encoding segment. Such vectors may be prepared by means of standard recombinant techniques well known in the art. See for example, see Ausbel (1992); Sambrook and Russell (2001); and U.S. Pat. No. 5,837,492.

Large amounts of the protein of the present invention may be produced by (a) expression in a suitable host or transgenic animal or (b) chemical synthesis using techniquest well known in the art. Proteins acids made by either of these techniques are also referred to as synthetic proteins herein. Expression vectors may include, for example, an origin of replication or autonomously replicating sequence (ARS) and expression control sequences, a promoter, an enhancer and necessary processing information sites, such as ribosome-binding sites, RNA splice sites, polyadenylation sites, transcriptional terminator sequences, and MRNA stabilizing sequences. Secretion signals may also be included where appropriate which allow the protein to cross and/or lodge in cell membranes, and thus attain its functional topology, or be secreted from the cell. Such vectors may be prepared by means of standard recombinant techniques well known in the art. See for example, see Ausbel (1992); Sambrook and Russell (2001); and U.S. Pat. No. 5,837,492.

The γ-carboxylase of the present invention is isolated following expression in a suitable host or chemical synthesis using techniques well known in the art. The isolated γ-carboxylase of the present invention is used to γ-carboxylate γ-carboxylated proteins, such as conantokins and other vitamin K-dependent proteins. The γ-carboxylase is contacted with the pro-protein which contains the γ-carboxylation recognition sequence and allowed to γ-carboxylate the protein. The γ-carboxylated protein is isolated and purified using techniques well known in the art.

The nucleic acid encoding γ-carboxylase of the present invention is used to γ-carboxylate γ-carboxylated proteins, such as conantokins and other vitamin K-dependent proteins, in vivo using techniques well known in the art. In one embodiment, a suitable host is prepared which contains an expression vector containing a γ-carboxylase encoding nucleic acid of the present invention and an expression vector containing a nucleic acid encoding a γ-carboxylated protein, such conantokin and other vitamin K-dependent protein. Nucleic acids encoding conantokins are well known in the art. See U.S. Pat. No. 6,172,041. Nucleic acids encoding other vitamin K-dependent proteins are also well known in the art. In a second embodiment, a suitable host is prepared which contains an expression vector containing a γ-carboxylase encoding nucleic acid and a nucleic acid encoding a γ-carboxylated protein, such conantokin and other vitamin K-dependent protein. In either embodiment, the host cells are grown under conditions suitable for growth and expression of the γ-carboxylase and the γ-carboxylated protein. The γ-carboxylase acts on the γ-carboxylated protein in vivo to properly γ-carboxylate the Glu residues in the protein.

### EXAMPLES

The present invention is described by reference to the following Examples, which are offered by way of illustration and are not intended to limit the invention in any manner. Standard techniques well known in the art or the techniques specifically described below were utilized.

### Example 1

### In Vitro γ-Carboxylation of ConG with γ-Carboxylase

In order to ascertain the fidelity of γ-carboxylation, it is essential to determine if the appropriate Glu residues are being modified and if the modification goes to completion. ConG, −20pro-ConG and pro-ConG are used as substrates to determine fidelity of carboxylation in vitro with the Conus γ-carboxylase produced in accordance with the present invention as described in U.S. Pat. No. 6,197,535. The identification of Gla by routine amino acid sequencing is not efficient due to poor recovery of Gla residues. In the present modified method, Gla-containing peptides are decarboxylated by heating under vacuum. The Gla residues are converted to Glu which can then be sequenced. If ¹⁴CO₂ is incorporated in the γ-carboxylation reaction, half of the molecules in the decarboxylated product will contain ¹⁴CO₂ covalently linked to the γ-C of modified Glu residues. Thus, by monitoring the radioactivity recovered at each step of the sequencing reaction, the position of Gla residues can be determined.

Products of the γ-carboxylation reaction are purified using a Waters Oasis [symbol] HLB Extraction Cartridge followed by reversed phase HPLC using Vydac C₁₈ column. The [¹⁴C]-containing fractions are dried and sequenced chemically with concomitant determination of radioactivity at each position in the sequence. When −20.pro-ConG and pro-ConG are used as the substrate, the radioactivity-containing fraction from the reversed phase HPLC column are dried and digested with endoproteinase Lys C. This is done to reduce the length of the peptide for sequencing without interfering with the identification of the γ-carboxylated residues. The Lys C digest is purified using a Waters Oasis [symbol] HLB Extraction Cartridge followed by reversed phase HPLC using Vydac C₁₈ column. The radioactivity eluted as a single peak coincidental with the A₂₂₀ peak. The chemical sequence of the material had the expected sequence.

The Gla determinations are carried out on a mixture of unmodified and variously modified substrate molecules. On the basis of these experiments, it is not possible to assign Gla residues to individual post-translationally modified substrate molecules. However, an average picture emerged. As in the case of the native product, Glu² is not carboxylated. The rest of the Glu residues are carboxylated.

### Example 2

### Cloning of Conus Textile γ-Carboxylase cDNA

The full-length C. textile γ-carboxylase cDNA was isolated by reverse transcription-PCR (RT-PCR) of venom duct RNA, using primers designed from conserved regions of mammalian γ-carboxylase proteins. A number of different internal primer sets were utilized to generate overlapping segments of the C. textile γ-carboxylase sequence. Most of the internal region of the C. textile cDNA was obtained in this manner, generating sequence to within ˜100 amino acids of the putative N- and C-termini of the protein. To obtain the ends of the cDNA sequence, nested PCR primers based on the C. textile cDNA sequence were used in 5′ and 3′ RACE to identify the transcription start site and poly A termination site, respectively. Merging of these overlapping segments generated a ˜2460 bp cDNA sequence with a single long open reading frame encoding a protein of 799 amino acids. This C. textile protein has substantial homology to the mammalian γ-carboxylase. Overall homology of the Conus sequence to the mammalian enzymes is ˜50%, although distinct regions within the protein show substantially higher and lower levels of sequence conservation. The RT-PCR with degenerate primers consistently identified the sequence presented here, with no evidence for any other related γ-carboxylase isoform being expressed in the C. textile venom duct. Much of the degenerate PCR used to initially clone the C. textile sequence employed non-proofreading thermostable polymerases and many cycles of PCR, both conditions that could introduce minor sequence errors. To obtain an accurate sequence, primers designed to the very most 5′ and 3′ ends of the C. textile cDNA sequence were used to amplify the full-length 2460 bp cDNA using a proofreading thermostable polymerase mixture and a minimal number of PCR cycles. This Long, Accurate-PCR generated the predicted 2460 bp product in good yield, with no evidence for alternative-splice isoforms of the C. textile γ-carboxylase mRNA being expressed in the venom duct. The full-length cDNA product was cloned and completely sequenced on both strands, yielding the sequence presented here.

The C. textile γ-carboxylase cDNA has a short 5′ untranslated region of ˜50 bp, and the first ATG start codon encountered initiates the long open reading frame encoding the γ-carboxylase protein. The cDNA sequence obtained by 3′ RACE terminates in a poly A tail, and this is preceded by a typical poly A addition signal (AATAA). An unusual feature is that the open reading frame lacks a typical stop codon (TAG, TAG, TGA) and instead continues into the poly A tail. We considered the possibility that the 3′ RACE technique had not identified the true 3′ end of the mRNA, or that a sequencing error obscured a stop codon that is present, but various experiments tend to rule this out. A variety of 3′ RACE experiments have been performed, using different PCR primers and cDNA preparations with different 3′ adapters, yet all of these experiments consistently identify the same 3′ poly A site. Numerous different PCR product clones representing this 3′ region have been thoroughly sequenced, and there are no ambiguities on the sequencing chromatograms that would suggest a sequencing anomaly that could change the open reading frame.

Finally, 3′ RACE was used to isolate the corresponding region of γ-carboxylase cDNA from the venom duct RNA of two other Conus species, omaria and episcopatus, that are snail-hunting species related to textile. The 3′ RACE identified poly A sites at essentially the same location in all three different species. The DNA sequences (and corresponding protein sequence) were highly homologous between the three species, but there is sequence variation as expected between species. Even though the sequence varies between the species, in all three the open reading frame lacks a typical stop codon and extends into the poly A tail. This suggests that our initial finding was not just a cloning or sequence artifact restricted to C. textile, but is a conserved feature of the γ-carboxylase mRNA across related Conus species. It is possible that Conus recognizes an atypical triplet as a termination codon, or that post-translation processing generates the true C-terminus of the Conus γ-carboxylase enzyme. Assuming that it terminates at the poly A tail, the size of the Conus γ-carboxylase protein that we have identified (799 amino acids) is very similar to that of the mammalian γ-carboxylase enzymes, so the overall size of the proteins appears to be conserved.

The nucleic acid sequence (SEQ ID NO:1 or 3) and amino acid sequence (SEQ ID NO:2 or 4) for C. textile γ-carboxylase are set forth in Tables 1 and 2, respectively. The 3′ nucleic acid sequence (SEQ ID NO:5 or 7) and C-terminal amino acid sequence (SEQ ID NO:6 or 8) for C. omaria are set forth in Tables 3 and 4, respectively. The 3′ nucleic acid sequence (SEQ ID NO:9 or 11) and C-terminal amino acid sequence (SEQ ID NO:10 or 12) for C. episcopatus are set forth in Tables 5 and 6, respectively.

The sequences for C. omaria and C. episcopatus represent about 200-220 amino acids at the C-terminus, starting at a position corresponding to approximately 590 of C. textile. Both of these sequences have several base pair and amino acid changes compared to the C. textile sequence as is expected for species homologs, although the sequence identity is quite high. All three species have the poly A tail in roughly the same location (the C. textile sequence is about 30 bp longer) and none of the three species has a typical stop codon.

TABLE 1
Nucleic Acid Sequence of C. textile γ-Carboxylase

| | |
|---|---|
| ATCTTTGTGAGCGTGATCCATCGCACAAACCATGCAAAGGCCAGGCAAGAAAGTGGCTGCTGATTCAGAG | (SEQ ID NO: 1) |
| GAATCAAATGACATCAGCCAACAAGCAGAAAACAGAGACCAGCTCCTCCCCCAGGAAGCCAGTCCCAAAG | |
| CGTGTGAGGAAGAGGACACAGAGGATGAAGAGGAAGAAGAGGACAAGTTCTACAAACTCTTTGGTTTCAG | |
| CTTGAGCGACCTCAAGTCATGGGACAGCTTTGTTCGTCTGTTGTCGCGCCCCGCTGACCCTGCCGGTCTG | |
| GCTTATATCCGTGTCACTTATGGGTTTTTGATGATGTGGGACGTGTTTGAGGAAAGGGGCCTGTCCCGTG | |
| CAGATATGCGATGGGGTGATGATGAGGCATGCAGGTTTCCTCTCTTCGACTTCATGCAACCCTTGCCCCT | |
| GCACATGATGGTCCTGCTGTACCTGATCATGCTGATTGGAACAGGAGGAATTCTATTAGGAGCCAAGTAC | |
| CGTGTGTGCTGCGTTATGCACCTGCTGCCCTACTGGTACATAGTGCTTCTGGACGAGTGCAGTTGGAACA | |
| ATCACTCCTATCTGTTTGGTCTCCTCTCTTTCCTCCTTCTGCTTTGCGATGCTAACCACTACTGGTCCAT | |
| GGACGGTCTGTTCAATGCCAAGGTTCGAAATACCGATGTTCCCTTGTGGAACTACACCCTCCTACGTACA | |
| CAGGTGTTTCTGGTGTACTTTTTGGCTGGGCTGAAGAAACTGGACATGGACTGGATCGCTGGTTACTCCA | |
| TGGGCCCTTTGAGTGATCATTGGGTCTTTTACCCGTTTACGTTCCTGATGACAGAAGACCAGGTGAGTGT | |
| GCTTGTGGTCCACCTGGGTGGACTTGCCATTGACTTGTTCGTCGGCTACCTGCTCTTCTTTGACAAGACA | |
| CCACCGATCGGTGTCATTATCAGTTCGTCATTCCACCTGATGAATGCACAGATGTTCAGCATAGGAATGT | |
| TTCCCTATGCCATGTTGGGTTTGACGCCTGTGTTCTTCTATGCCAACTGGCCGAGGGCCCCGTTTCGCCG | |
| CATTCCACGATCCTTGAGGATTCTTACCCCTGATGATGGAGAGGATGATACGCTGCCTTCGGAGAAGTGC | |
| TTATACACAAAAGAACAGGCCAAACCAGAACTGGCCAGCACCCCTGAGCATGAAAACACTGCAGTCCGCA | |
| AACAGTTGACACCACCCACTCAGCCCACGTTCCGGCATCATGCTGCCGCTGCCTTCACCGTTTTCTTCAT | |
| TCTGTGGCAGATGTTTTTGCCTTTCTCTCATTTTATCACAAAGGGCAACAACAGCTGGACCCAGGGACTC | |
| TACGGCTACTCCTGGGACATGATGGTTCACACCCGCAGCACTCAGCACACCAGGATCTCCTTCATCAACA | |
| AGGACACAGGAGAGCGAGGGTTCCTGGACCCGCAGGCATGGAGCAAGTCACATCGATGGGCGCATAACGC | |
| TAAGATGATGAAGCAGTACGCCAGGTGCATCGCTCGCCGACTGAAGAAGCATGAAATCGACAATGTGGAA | |
| ATCTATTTTGATGTCTGGATATCTCTGAATCATCGCTTCCAGCAACGGATCGTGAACCCCAATGTGGACA | |
| TTTTAACAGCCGAATGGAGTGTCTTTAAGTCCACTCCATGGATGATGCCCTTGCTGGTCGACTTGTCTAA | |
| TTGGCGAAGCAAGTTGAAAGAGATTGAGGACGACATTTTCAACTCAACCGACCTGTATGAAATAGTCTTT | |
| CTGGCTGACTTTCCTGGTTTGTACCTGGAGAACTTTGTCCACGGCAGCGTCGGGAGTCTCAACATCTCTG | |
| TACTGCAGGGCCAGGTGGTGGTGGAGGTGCTTCCAGAGGAGGACAGTCTAGAAGAGCCCTACAACATCAG | |
| CATCAGTGATGGCCAAGAGTCATTGATTCCCACAGGGGTGTTCCACAAGGTGTACACAGTGTCTGAAGTG | |
| CCCTCCTGTTACATGTACATCTACATGGTCACGGAAGAGACAGAGTTCCTTGAGAAACTCAAAGAGCTGG | |
| AACACGCCCTCAACGGCTCCCTGGATGCTCCAGTTCCAGACAAGTTTGCCGAAGATCCTAAACTTGATCA | |
| GTATATGGAGGTACTCAAAACGAAGAATGCAACTCCACCACCAACCTCTCAAGAGGAGCAAAGTTTCATA | |
| CAGCTGTTTATGAGTTTTCTGAAAATGCATTATATGTCTATGTATCGTGGACTGCAGCTGATAAAAGGCG | |
| CCATGTGGTCCATGTACTCCGGGGAATCTTACCGAGAGTTCTTGAAGAAACTGGAGCTACAGAAAATGCT | |
| GGCGGAGAATGCCACCCTGGTGGCAAACGCCACCCAAGGGGTGAATAACACCCAGACGATGAACAACACC | |
| TTGAACAACACCAAGGAGAAAGACAACACCCAAAGGGTTAACAAACCG | |
| ATGCAAAGGCCAGGCAAGAAAGTGGCTGCTGATTCAGAGGAATCAAATGACATCAGCCAACAAGCAGAAA | (SEQ ID NO: 3) |
| ACAGAGACCAGCTCCTCCCCCAGGAAGCCAGTCCCAAAGCGTGTGAGGAAGAGGACACAGAGGATGAAGA | |
| GGAAGAAGAGGACAAGTTCTACAAACTCTTTGGTTTCAGCTTGAGCGACCTCAAGTCATGGGACAGCTTT | |
| GTTCGTCTGTTGTCGCGCCCCGCTGACCCTGCCGGTCTGGCTTATATCCGTGTCACTTATGGGTTTTTGA | |
| TGATGTGGGACGTGTTTGAGGAAAGGGGCCTGTCCCGTGCAGATATGCGATGGGGTGATGATGAGGCATG | |
| CAGGTTTCCTCTCTTCGACTTCATGCAACCCTTGCCCCTGCACATGATGGTCCTGCTGTACCTGATCATG | |
| CTGATTGGAACAGGAGGAATTCTATTAGGAGCCAAGTACCGTGTGTGCTGCGTTATGCACCTGCTGCCCT | |
| ACTGGTACATAGTGCTTCTGGACGAGTGCAGTTGGAACAATCACTCCTATCTGTTTGGTCTCCTCTCTTT | |
| CCTCCTTCTGCTTTGCGATGCTAACCACTACTGGTCCATGGACGGTCTGTTCAATGCCAAGGTTCGAAAT | |
| ACGGATGTTCCCTTGTGGAACTACACCCTCCTACGTACACAGGTGTTTCTGGTGTACTTTTTGGCTGGGC | |
| TGAAGAAACTGGACATGGACTGGATCGCTGGTTACTCCATGGGCCGTTTGAGTGATCATTGGGTCTTTTA | |
| CCCGTTTACGTTCCTGATGACAGAAGACCAGGTGAGTGTGCTTGTGGTCCACCTGGGTGGACTTGCCATT | |
| GACTTGTTCGTGGGCTACCTGCTCTTCTTTGACAAGACACGACCGATCGGTGTCATTATCAGTTCGTCAT | |
| TCCACCTGATGAATGCACAGATGTTCAGCATAGGAATGTTTCCGTATGCCATGTTGGGTTTGACGCCTGT | |
| GTTCTTCTATGCCAACTGGCCGAGGGCCCTGTTTCGCCGCATTCCACGATCCTTGAGGATTCTTACCCCT | |
| GATGATGGAGAGGATGATACGCTGCCTTCGGAGAAGTGCTTATACACAAAAGAACAGGCCAAACCAGAAC | |
| TGGCCAGCACCCCTGAGCATGAAAACACTGCAGTCCGCAAACAGTTGACACCACCCACTCAGCCCACGTT | |
| CCGGCATCATGCTGCCGCTGCCTTCACCGTTTTCTTCATTCTGTGGCAGATGTTTTTGCCTTTCTCTCAT | |
| TTTATCACAAAGGGCAACAACAGCTGGACCCAGGGACTCTACGGCTACTCCTGGGACATGATGGTTCACA | |
| CCCGCAGCACTCAGCACACCAGGATCTCCTTCATCAACAAGGACACAGGAGAGCGAGGGTTCCTGGACCC | |
| GCAGGCATGGAGCAAGTCACATCGATGGGCGCATAACGCTAAGATGATGAAGCAGTACGCCAGGTGCATC | |
| GCTCGCCGACTGAAGAAGCATGAAATCGACAATGTGGAAATCTATTTTGATGTCTGGATATCTCTGAATC | |
| ATCGCTTCCAGCAACGGATCGTGAACCCCAATGTGGACATTTTAACAGCCGAATGGAGTGTCTTTAAGTC | |
| CACTCCATGGATGATCCCCTTGCTGGTCGACTTGTCTAATTGGCGAAGCAAGTTGAAAGAGATTGAGGAC | |
| GACATTTTCAACTCAACCGACCTGTATGAAATAGTCTTTCTGGCTGACTTTCCTGGTTTGTACCTGGAGA | |
| ACTTTGTCCACGGCAGCGTCGGGAGTCTCAACATCTCTGTACTGCAGGGCCAGGTGGTGGTGGAGGTGCT | |
| TCCAGAGGAGGACAGTCTAGAAGAGCCCTACAACATCAGCATCAGTGATGGCCAAGAGTCATTGATTCCC | |
| ACAGGGGTGTTCCACAAGGTGTACACAGTGTCTGAAGTGCCCTCCTGTTACATGTACATCTACATGGTCA | |
| CGGAAGAGACAGAGTTCCTTGAGAAACTCAAAGAGCTGGAACACGCCCTCAACGGCTCCCTGGATGCTCC | |
| AGTTCCAGACAAGTTTGCCGAAGATCCTAAACTTGATCAGTATATGGAGGTACTCAAAACGAAGAATGCA | |
| ACTCCACCACCAACCTCTCAAGAGGAGCAAAGTTTCATACAGCTGTTTATGAGTTTTCTGAAAATGCATT | |
| ATATGTCTATGTATCGTGGACTGCAGCTGATAAAAGGCGCCATGTGGTCCATGTACTCCGGGGAATCTTA | |
| CCGAGAGTTCTTGAAGAAACTGGAGCTACAGAAAATGCTGGCGGAGAATGCCACCCTGGTGGCAAACGCC | |
| ACCCAAGGGGTGAATAACACCCAGACGATGAACAACACCTTGAACAACACCAAGGAGAAAGACAACACCC | |
| AAAGGGTTAACAAACCGCAGGAAAAGAAGGCCCCCCAGAAGGCAGACAGCCCCTAACAGCATCTCTGCAG | |
| AATGAGGGCGTCATGCCTCTGTTCTGCATTGTAAATCTTCAATGTCAGACTCGCTGTCATGAGTCAGGAT | |
| GCCAAGGGTTGATTCTAAATGAAAAAA | |

TABLE 2
Protein Sequence of C. textile γ-Carboxylase

| | |
|---|---|
| MQRPGKKVAADSEESNDISQQAENRDQLLPQEASPKACEEEDTEDEEEEEDKFYKLFGFSLSDLKSWDSF | (SEQ ID NO: 2) |
| VRLLSRPADPAGLAYIRVTYGFLMMWDVFEERGLSRADMRWGDDEACRFPLFDFMQPLPLHMMVLLYLIM | |
| LIGTGGILLGAKYRVCCVMHLLPYWYIVLLDECSWNNHSYLFGLLSFLLLLCDANHYWSMDGLFNAKVRN | |
| TDVPLWNYTLLRTQVFLVYFLAGLKKLDMDWIAGYSMGRLSDHWVFYPFTFLMTEDQVSVLVVHLGGLAI | |
| DLFVGYLLFFDKTPPIGVIISSSFHLMNAQMFSIGMFPYAMLGLTPVFFYANWPRAPFRRIPRSLRILTP | |
| DDGEDDTLPSEKCLYTKEQAKPELASTPEHENTAVRKQLTPPTQPTFRHHAAAAFTVFFILWQMFLPFSH | |
| FITKGNNSWTQGLYGYSWDMMVHTRSTQHTRISFINKDTGERGFLDPQAWSKSHRWAHNAKMMKQYARCI | |
| ARRLKKHEIDNVEIYFDVWISLNHRFQQRIVNPNVDILTAEWSVFKSTPWMMPLLVDLSNWRSKLKEIED | |
| DIFNSTDLYEIVFLADFPGLYLENFVHGSVGSLNISVLQGQVVVEVLPEEDSLEEPYNISISDGQESLIP | |
| TGVFHKVYTVSEVPSCYMYIYMVTEETEFLEKLKELEHALNGSLDAPVPDKFAEDPKLDQYMEVLKTKNA | |
| TPPPTSQEEQSFIQLFMSFLKMHYMSMYRGLQLIKGAMWSMYSGESYREFLKKLELQKMLAENATLVANA | |
| TQGVNNTQTMNNTLNNTKEKDNTQRVNKP | |
| MQRPGKKVAADSEESNDISQQAENRDQLLPQEASPKACEEEDTEDEEEEEDKFYKLFGFSLSDLKSWDSF | (SEQ ID NO: 4) |
| VRLLSRPADPAGLAYIRVTYGFLMMWDVFEERGLSRADMRWGDDEACRFPLFDFMQPLPLHMMVLLYLIM | |
| LIGTGGILLGAKYRVCCVMHLLPYWYIVLLDECSWNNHSYLFGLLSFLLLLCDANHYWSMDGLFNAKVRN | |
| TDVPLWNYTLLRTQVFLVYFLAGLKKLDMDWIAGYSMGRLSDHWVFYPFTFLMTEDQVSVLVVHLGGLAI | |
| DLFVGYLLFFDKTRPIGVIISSSFHLMNAQMFSIGMFPYAMLGLTPVFFYANWPRALFRRIPRSLRILTP | |
| DDGEDDTLPSEKCLYTKEQAKPELASTPEHENTAVRKQLTPPTQPTFRHHAAAAFTVFFILWQMFLPFSH | |
| FITKGNNSWTQGLYGYSWDMMVHTRSTQHTRISFINKDTGERGFLDPQAWSKSHRWAHNAKMMKQYARCI | |
| ARRLKKHEIDNVEIYFDVWISLNHRFQQRIVNPNVDILTAEWSVFKSTPWMMPLLVDLSNWRSKLKEIED | |
| DIFNSTDLYEIVFLADFPGLYLENFVHGSVGSLNISVLQGQVVVEVLPEEDSLEEPYNISISDGQESLIP | |
| TGVFHKVYTVSEVPSCYMYIYMVTEETEFLEKLKELEHALNGSLDAPVPDKFAEDPKLDQYMEVLKTKNA | |
| TPPPTSQEEQSFIQLFMSFLKMHYMSMYRGLQLIKGAMWSMYSGESYREFLKKLELQKMLAENATLVANA | |
| TQGVNNTQTMNNTLNNTKEKDNTQRVNKPQEKKAPQKADSP | |

TABLE 3
3′ Nucleic Acid Sequence of C. omaria γ-Carboxylase

| | |
|---|---|
| GGGAGTCTCAACATCTCTGTACTGCAGGGGCAGGTGGTGGTGGAGGTGCTTCCAGAGGAGGACAGTCTAG | (SEQ ID NO: 5) |
| AAAAACCCTACAACATCAGCATCAATGATGGCCACGAGTCATTGATTCCCACAGGGGTATTCCACAAGGT | |
| GTACACAGTGTCTGAAGTGCCCTCCTGTTACATGTACATCTACATGGTCACGGAAGAGACGGAGTTCTTT | |
| GAGAACGTCAAAGAGCTGGAACACGCCCTCAACGGCTCCCTGGATGCTCCAGTTCCAGACAAGTTTGCCA | |
| AAGATCCTAAACTTGATCAATATATGGAGCTACTCAAAGTGAAGAATGCAGCTCCACCACCGGCCCCTCG | |
| AGCGGAGAGAAGTTTCATAGAGCTGTTTATGAGTTTTCTGAAAATGCATTATATGTCTATGTATCGTGGA | |
| CTGCAGCTGATAAAAGGCGCCGTGTGGTCCATGTACTCTGGGGAATCTTACCGAGAGTACCTGAAGGAAC | |
| TGGAATTACAGGCAATGCTGGGGGAGAATGCCACCCTGGTGGCAAACGCCACCCAAGGGGTGAATAACAC | |
| CCAGACGATGAACAACACCTTATTGAACAACACCAAAAAAAAAAAAAAAAAA | |
| GGGAGTCTCAACATCTCTGTACTGCAGGGCCAGGTGGTGGTGGAGGTGCTTCCAGAGGAGGACAGTCTAG | (SEQ ID NO: 7) |
| AAAAACCCTACAACATCAGCATCAATGATGGCCACGAGTCATTGATTCCCACAGGGGTATTCCACAAGGT | |
| GTACACAGTGTCTGAAGTGCCCTCCTGTTACATGTACATCTACATGGTCACGGAAGAGACGGAGTTCTTT | |
| GAGAACGTCAAAGAGCTGGAACACGCCCTCAACGGCTCCCTGGATGCTCCAGTTCCAGACAAGTTTGCCA | |
| AAGATCCTAAACTTGATCAATATATGGAGGTACTCAAAGTGAAGAATGCAGCTCCACCACCGGCCCCTCG | |
| AGCGGAGAGAAGTTTCATAGAGCTGTTTATGAGTTTTCTGAAAATGCATTATATGTCTATGTATCGTGGA | |
| CTGCAGCTGATAAAAGGCGCCGTGTGGTCCATGTACTCTGGGGAATCTTACCGAGAGTACCTGAAGGAAC | |
| TGGAATTACAGGCAATGCTGGGGGAGAATGCCACCCTGGTGGCAAACGCCACCCAAGGGGTGAATAACAC | |
| CCAGACGATGAACAACACCTTATTGAACAACACCAAGGAGAAAAACAACACCCAAAGGGTTAACAAGCCG | |
| CAGGAAAAGAAGGCCCCCCAGAAGGCAGACAGCCCCTAACAGCATCTCTGCAGAATGAGGGCATCATGCC | |
| TCTGTTCTGCATTTTAAATCTTCGATGTCAGACACGCTGTCATGAGTCAGGATGCCAAGGGTTGATTCTA | |
| AATGAAAAAA | |

TABLE 4
C-Terminal Protein Sequence of C. omaria γ-Carboxylase

| | |
|---|---|
| GSLNISVLQGQVVVEVLPEEDSLEKPYNISINDGHESLIPTGVFHKVYTVSEVPSCYMYIYMVTEETEFF | (SEQ ID NO: 6) |
| ENVKELEHALNGSLDAPVPDKFAKDPKLDQYMEVLKVKNAAPPPAPRAERSFIELFMSFLKMHYMSMYRG | |
| LQLIKGAVWSMYSGESYREYLKELELQAMLGENATLVANATQGVNNTQTMNNTLLNNTKKKKKK | |
| GSLNISVLQGQVVVEVLPEEDSLEKPYNISINDGHESLIPTGVFHKVYTVSEVPSCYMYIYMVTEETEFF | (SEQ ID NO: 8) |
| ENVKELEHALNGSLDAPVPDKFAKDPKLDQYMEVLKVKNAAPPPAPRAERSFIELFMSFLKMHYMSMYRG | |
| LQLIKGAVWSMYSGESYREYLKELELQAMLGENATLVANATQGVNNTQTMNNTLLNNTKEKNNTQRVNKP | |
| QEKKAPQKADSP | |

TABLE 5
3′ Nucleic Acid Sequence of C. episcopatus γ-Carboxylase

| | |
|---|---|
| GGGAGTCTCAACATCTCTGTACTGCAGGGCCAGGTGGTGGTGGAGGTGCTTCCAGAGGAGGACAGTCTAG | (SEQ ID NO: 9) |
| AACAGCCCTACAACATCAGCATCAGTGATGGCCACGAGTCATTGATTCCCACAGGGGTGTTCCACAAGGT | |
| GTACACAGTGTCTGAAGTGCCCTCCTGTTACATGTACATCTAGATGGTCACGGAAGAGACGGAGTTCTTT | |
| GAGAACCTCAAAGAGCTGGAACACGCCCTCAACGGCTCCCTGGATGCTCCAGTACCAGACAAGTTTGCCA | |
| AAGATCCTAAACTTGATCAATATATGGAGGTACTCAAGGTGAAGAATGCAGCTCCACCACCGGCCCCTCC | |
| AGCGGACAGAAGTTTCATACAGCTGTTTATGAGTTTTCTGAAAATGCATTATATGTCTATGTATCGTGGA | |
| CTGCAGCTGATAAAAGGCGCCGTGTGGTCCATGTACTCTGGGGAATCTTACCGAGAGTACCTGAAGGAAC | |
| TGGAGCTACAGGCAATGCTGGGGGAGAATGTCACCCTGGTGGCAAATGCCACCGAAGGGGTGAATAAAAC | |
| CCAGATGATGAACAACACCTTGAACAACACCAAAAAAAAAAAAAAAAAA | |
| GGGAGTCTCAACATCTCTGTACTGCAGGGCCAGGTGGTGGTGGAGGTGCTTCCAGAGGAGGACAGTCTAG | (SEQ ID NO: 11) |
| AACAGCCCTACAACATCAGCATCAGTGATGGCCACGAGTCATTGATTCCCACAGGGGTGTTCCACAAGGT | |
| GTACACAGTGTCTGAAGTGCCCTCCTGTTACATGTACATCTACATGGTCACGGAAGAGACGGAGTTCTTT | |
| GAGAACCTCAAAGAGCTGGAACACGCCCTCAACGGCTCCCTGGATGCTCCAGTACCAGACAAGTTTGCCA | |
| AAGATCCTAAACTTGATCAATATATGGAGGTACTCAAAGTGAAGAATGCAGCTCCACCACCGGCCCCTCC | |
| AGCGGACAGAAGTTTCATACAGCTGTTTATGAGTTTTCTGAAAATGCATTATATGTCTATGTATCGTGGA | |
| CTGCAGCTGATAAAAGGCGCCGTGTGGTCCATGTACTCTGGGGAATCTTACCGAGAGTACCTGAAGGAAC | |
| TGGAGCTACAGGCAATGCTGGGGGAGAATGTCACCCTGGTGGCAAATGCCACCCAAGGGGTGAATAAAAC | |
| CCAGATGATGAACAACACCTTGAACAACACCAAGGAGAAAAACAACACCCAAAGGGTTAACAAGCCGCAG | |
| GAAAAGAAGGCCCCCCAGAAGGCAGACAGCCCCTAACAGCATCTCTGCAGAATGAGGGCATCATGCCTCT | |
| GTTCTGCATTTTAAATCTTCAATGTCAGACACGCTGTCATGAGTCAGGATGCCAAGGGTTGATTCTAAAT | |
| GAAAAAA | |

TABLE 6
C-Terminal Protein Sequence of C. episcopatus γ-Carboxylase

| | |
|---|---|
| GSLNISVLQGQVVVEVLPEEDSLEQPYNISISDGHESLIPTGVFHKVYTVSEVPSCYMYIYMVTEETEFF | (SEQ ID NO: 10) |
| ENLKELEHALNGSLDAPVPDKFAKDPKLDQYMEVLKVKNAAPPPAPPADRSFIQLFMSFLKMHYMSMYRG | |
| LQLIKGAVWSMYSGESYREYLKELELQAMLGENVTLVANATQGVNKTQMMNNTLNNTKKKKKK | |
| GSLNISVLQGQVVVEVLPEEDSLEQPYNISISDGHESLIPTGVFHKVYTVSEVPSCYMYIYMVTEETEFF | (SEQ ID NO: 12) |
| ENLKELEHALNGSLDAPVPDKFAKDPKLDQYMEVLKVKNAAPPPAPPADRSFIQLFMSFLKMHYMSMYRG | |
| LQLIKGAVWSMYSGESYREYLKELELQAMLGENVTLVANATQGVNKTQMMNNTLNNTKEKNNTQRVNKPQ | |
| EKKAPQKADSP | |

### Example 3

### Expression of γ-Carboxylase in Host Cells

The C. textile γ-carboxylase cDNA sequence is cloned and expressed as described by Walker et al. (2001). Briefly, the cDNA coding sequence is cloned in frame with green fluorescent protein (GFP) in the expression plasmid pRmHa-3.GFP (Walker et al., 2001). Expression in this plasmid is under control of the Drosophila inducible metallothionein promoter and carries the alcohol dehydrogenase poly (A) addition signal. Drosophila Schneider 2 (S2) cells are transfected with the resultant plasmid containing the Conus γ-carboxylase coding sequence using CelIFECTIN™ (Life Technologies). Twenty-four hours after transfection, cells are induced with 0.7 mM CuSO₄. Forty-eight hours after transfection, cells are found to express GFP as seen by fluorescent microscopy. The plasmid containing the Conus γ-carboxylase coding sequence and the GFP sequence is modified to add a stop codon at the end of the Conus γ-carboxylase coding sequence and to delete the GFP coding sequence.

This modified expression vector and a vector DNA expressing the hygromyocin gene are used to cotransfect Drosophila S2 cells. Hygromyocin resistant cells are selected and individual clones are expanded. The expanded clones are analyzed for expression of Conus γ-carboxylase. Briefly, the cells are induced with 0.7 mM CuSO₄ and harvested 48 hours after induction. Cells are washed twice with phosphate-buffered saline and resuspended in buffer containing 25 MM 4-morpholinepropanesulfonic acid, Ph7.0, 0.5 M NaCl, 0.2% 3-[(3-chloramidopropyl)dimethyl-ammonio]-1-propane sulfonic acid/poshphatidyl choline, 2 MM EDTA, 2 MM dithiothreitol, 0.2 μg/ml leupeptin, 0.8 μg/ml pepstatin and 0.04 Mg/ml phenylmethylsulfonyl fluoride. The cell suspension is briefly sonicated and incubated in ice for 20 min. The lysate is assayed for Conus γ-carboxylase activity as described in Example 1. The isolated Conus γ-carboxylase is found to be biologically active and to properly γ-carboxylate ConG, i.e. Glu² is not γ-carboxylated while the remaining Glu residues are γ-carboxylated. The cells expressing the Conus γ-carboxylase are grown and maintained.

### Example 4

### Synthesis of γ-Carboxylated ConG in Host Cells

The cDNA sequence coding for the ConG propeptide (U.S. Pat. No. 6,172,041) is cloned and expressed as described by Walker et al. (2001). Briefly, the cDNA for the ConG propeptide sequence is cloned into pRmHa-3.GFP under control of the Drosophila metallothionenin promoter as described in Example 3. The resultant plasmid is modified to insert a stop codon and to delete the GFP coding sequence as described in Example 3. This expression vector is used to transfect cells expressing γ-carboxylase prepared in Example 3. Cells expressing γ-carboxylase and ConG propeptide are selected and expanded. ConG is isolated from these cells and analyzed for proper γ-carboxylation as described in Example 1. The Glu residues in ConG are found to be properly γ-carboxylated, i.e. Glu² is not γ-carboxylated while the remaining Glu residues are γ-carboxylated.

It will be appreciated that the methods and compositions of the instant invention can be incorporated in the form of a variety of embodiments, only a few of which are disclosed herein. It will be apparent to the artisan that other embodiments exist and do not depart from the spirit of the invention. Thus, the described embodiments are illustrative and should not be construed as restrictive.

### Bibliography

Ausubel, F. M., et al. (1992). Current Protocols in Molecular Biology and supplements, J. Wiley and Sons, New York.

Berkner, K. L. et al. (1992). Proc. Natl. Acad. Sci. USA 89: 6242-6246.

Cheung, A. et al. (1989). Arch. Biochem. Biophys. 274: 574-581.

Haack, J. A. et al. (1990). J. Biol. Chem. 265: 6025-6029.

Knobloch, J. E. and Suttie, J. W. (1987). J. Biol. Chem. 262: 15334-15337.

McIntosh, J. M. et al. (1984). J. Biol. Chem. 259: 14343-14346.

Olivera, B. M. et al. (1990). Science 249: 257-263.

Price, P. A. and Williamson, M. K. (1985). J. Biol. Chem. 260: 14971-14975.

Price, P. A. et al. (1987). Proc. Natl. Acad. Sci. USA 84: 8335-8339.

Rigby, A. C. et al. (1997). Biochemistry 36: 6906-6914.

Roth, D. A. et al. (1993). Proc. Natl. Acad. Sci. USA 90: 8372-8376.

Sambrook, J. and Russell, D. W. (2001). Molecular Cloning: A Laboratory Manual, 3^{rd} Ed., Cold Spring Harbor Laboratory Press, Cold Spring Harbor, N.Y.

Skjaebaek, N. et al. (1997). J. Biol. Chem. 272: 2291-2299.

Stanley, T. B. et al. (1997). FEBS Let.t 407: 85-88.

Stenflo, J. et al. (1974). Proc. Natl. Acad. Sci. USA 71: 2730-2733.

Ulrich, M. et al. (1988). J. Biol. Chem. 263: 9697-9702.

Walker, C. S. et al. (2001). J. Biol. Chem. 276:7769-7774.

Warder, S. E. et al. (1997). FEBS Lett. 411: 19-26.

White, H. S. et al. (1997). J Neurosci. Abst.

Wu, S.-M. et al. (1991a). Proc. Natl. Acad. Sci. USA 88: 2236-2240.

Wu, S.-M. et al. (1991b). Science 254: 1634-1636.

U.S. Pat. No. 5,837,492

U.S. Pat. No. 6,172,041

U.S. Pat. No. 6,197,535

What is claimed is:

1. A synthetic nucleic acid encoding a protein selected from the group consisting of a γ-carboxylase comprising an amino acid sequence as set forth in SEQ ID NO:2 or 4 and a protein having at least 95% identity to said γ-carboxylase and capable of γ-carboxylating Conantokin G.

2. The synthetic nucleic acid of claim 1, wherein said nucleic acid is selected from the group consisting of a nucleic acid which comprises a nucleotide sequence as set forth in SEQ ID NO:1 or 3 and a nucleic acid which comprises a nucleotide sequence having at least 95% identity to the nucleotide sequence set forth in SEQ ID NO:1 or 3.

3. A vector comprising the nucleic acid of claim 1.

4. The vector of claim 3 which is an expression vector.

5. A vector comprising the nucleic acid of claim 2.

6. The vector of claim 5 which is an expression vector.

7. Host cells containing the vector of claim 3.

8. Host cells containing the vector of claim 4.

9. Host cells containing the vector of claim 5.

10. Host cells containing the vector of claim 6.

11. The host cells of claim 8 which further comprises an expression vector comprising a nucleic acid sequence encoding a conantokin.

12. The host cells of claim 10 which further comprises an expression vector comprising a nucleic acid sequence encoding a conantokin.

13. A method for producing γ-carboxylase which comprises growing the host cells of claim 8 under conditions suitable for growth and isolating the expressed γ-carboxylase.

14. A method for producing γ-carboxylase which comprises growing the host cells of claim 10 under conditions suitable for growth and isolating the expressed γ-carboxylase.

15. A method for producing γ-carboxylated conantokin which comprises growing the host cells of claim 11 under conditions suitable for growth and isolating the γ-carboxylated conantokin.

16. A method for producing γ-carboxylated conantokin which comprises growing the host cells of claim 12 under conditions suitable for growth and isolating the γ-carboxylated conantokin.

17. An isolated γ-carboxylase selected from the group consisting of a γ-carboxylase comprising an amino acid sequence as set forth in SEQ ID NO:2 or 4 and a protein having at least 95% identity to said γ-carboxylase and capable of γ-carboxylating Cononatokin G.

18. A method for producing γ-carboxylated conantokin which comprises combining together a γ-carboxylase and a conantokin propeptide containing a Conus γ-carboxylase recognition sequence to produce a γ-carboxylated conantokin and isolating the γ-carboxylated conantokin, wherein said γ-carboxylase is selected from the group consisting of a γ-carboxylase comprising an amino acid sequence as set forth in SEQ ID NO:2 or 4 and a protein having at least 95% identity to said γ-carboxylase and capable of γ-carboxylating Conantokin G.


SEQUENCE LISTING (12 sequences)

SEQ ID NO: 1 - DNA, 2428 bp, Conus textile
  misc_feature (32)..(34): start codon
atctttgtgagcgtgatccatcgcacaaaccatgcaaaggccaggcaagaaagtggctgctgattcagaggaatcaaatgacatcagccaacaagcagaaaacagagaccagctcctcccccaggaagccagtcccaaagcgtgtgaggaagaggacacagaggatgaagaggaagaagaggacaagttctacaaactctttggtttcagcttgagcgacctcaagtcatgggacagctttgttcgtctgttgtcgcgccccgctgaccctgccggtctggcttatatccgtgtcacttatgggtttttgatgatgtgggacgtgtttgaggaaaggggcctgtcccgtgcagatatgcgatggggtgatgatgaggcatgcaggtttcctctcttcgacttcatgcaacccttgcccctgcacatgatggtcctgctgtacctgatcatgctgattggaacaggaggaattctattaggagccaagtaccgtgtgtgctgcgttatgcacctgctgccctactggtacatagtgcttctggacgagtgcagttggaacaatcactcctatctgtttggtctcctctctttcctccttctgctttgcgatgctaaccactactggtccatggacggtctgttcaatgccaaggttcgaaatacggatgttcccttgtggaactacaccctcctacgtacacaggtgtttctggtgtactttttggctgggctgaagaaactggacatggactggatcgctggttactccatgggccgtttgagtgatcattgggtcttttacccgtttacgttcctgatgacagaagaccaggtgagtgtgcttgtggtccacctgggtggacttgccattgacttgttcgtgggctacctgctcttctttgacaagacaccaccgatcggtgtcattatcagttcgtcattccacctgatgaatgcacagatgttcagcataggaatgtttccgtatgccatgttgggtttgacgcctgtgttcttctatgccaactggccgagggccccgtttcgccgcattccacgatccttgaggattcttacccctgatgatggagaggatgatacgctgccttcggagaagtgcttatacacaaaagaacaggccaaaccagaactggccagcacccctgagcatgaaaacactgcagtccgcaaacagttgacaccacccactcagcccacgttccggcatcatgctgccgctgccttcaccgttttcttcattctgtggcagatgtttttgcctttctctcattttatcacaaagggcaacaacagctggacccagggactctacggctactcctgggacatgatggttcacacccgcagcactcagcacaccaggatctccttcatcaacaaggacacaggagagcgagggttcctggacccgcaggcatggagcaagtcacatcgatgggcgcataacgctaagatgatgaagcagtacgccaggtgcatcgctcgccgactgaagaagcatgaaatcgacaatgtggaaatctattttgatgtctggatatctctgaatcatcgcttccagcaacggatcgtgaaccccaatgtggacattttaacagccgaatggagtgtctttaagtccactccatggatgatgcccttgctggtcgacttgtctaattggcgaagcaagttgaaagagattgaggacgacattttcaactcaaccgacctgtatgaaatagtctttctggctgactttcctggtttgtacctggagaactttgtccacggcagcgtcgggagtctcaacatctctgtactgcagggccaggtggtggtggaggtgcttccagaggaggacagtctagaagagccctacaacatcagcatcagtgatggccaagagtcattgattcccacaggggtgttccacaaggtgtacacagtgtctgaagtgccctcctgttacatgtacatctacatggtcacggaagagacagagttccttgagaaactcaaagagctggaacacgccctcaacggctccctggatgctccagttccagacaagtttgccgaagatcctaaacttgatcagtatatggaggtactcaaaacgaagaatgcaactccaccaccaacctctcaagaggagcaaagtttcatacagctgtttatgagttttctgaaaatgcattatatgtctatgtatcgtggactgcagctgataaaaggcgccatgtggtccatgtactccggggaatcttaccgagagttcttgaagaaactggagctacagaaaatgctggcggagaatgccaccctggtggcaaacgccacccaaggggtgaataacacccagacgatgaacaacaccttgaacaacaccaaggagaaagacaacacccaaagggttaacaaaccg

SEQ ID NO: 2 - protein, 799 aa, Conus textile
MQRPGKKVAADSEESNDISQQAENRDQLLPQEASPKACEEEDTEDEEEEEDKFYKLFGFSLSDLKSWDSFVRLLSRPADPAGLAYIRVTYGFLMMWDVFEERGLSRADMRWGDDEACRFPLFDFMQPLPLHMMVLLYLIMLIGTGGILLGAKYRVCCVMHLLPYWYIVLLDECSWNNHSYLFGLLSFLLLLCDANHYWSMDGLFNAKVRNTDVPLWNYTLLRTQVFLVYFLAGLKKLDMDWIAGYSMGRLSDHWVFYPFTFLMTEDQVSVLVVHLGGLAIDLFVGYLLFFDKTPPIGVIISSSFHLMNAQMFSIGMFPYAMLGLTPVFFYANWPRAPFRRIPRSLRILTPDDGEDDTLPSEKCLYTKEQAKPELASTPEHENTAVRKQLTPPTQPTFRHHAAAAFTVFFILWQMFLPFSHFITKGNNSWTQGLYGYSWDMMVHTRSTQHTRISFINKDTGERGFLDPQAWSKSHRWAHNAKMMKQYARCIARRLKKHEIDNVEIYFDVWISLNHRFQQRIVNPNVDILTAEWSVFKSTPWMMPLLVDLSNWRSKLKEIEDDIFNSTDLYEIVFLADFPGLYLENFVHGSVGSLNISVLQGQVVVEVLPEEDSLEEPYNISISDGQESLIPTGVFHKVYTVSEVPSCYMYIYMVTEETEFLEKLKELEHALNGSLDAPVPDKFAEDPKLDQYMEVLKTKNATPPPTSQEEQSFIQLFMSFLKMHYMSMYRGLQLIKGAMWSMYSGESYREFLKKLELQKMLAENATLVANATQGVNNTQTMNNTLNNTKEKDNTQRVNKP

SEQ ID NO: 3 - DNA, 2547 bp, Conus textile
  CDS (1)..(2433)
atgcaaaggccaggcaagaaagtggctgctgattcagaggaatcaaatgacatcagccaacaagcagaaaacagagaccagctcctcccccaggaagccagtcccaaagcgtgtgaggaagaggacacagaggatgaagaggaagaagaggacaagttctacaaactctttggtttcagcttgagcgacctcaagtcatgggacagctttgttcgtctgttgtcgcgccccgctgaccctgccggtctggcttatatccgtgtcacttatgggtttttgatgatgtgggacgtgtttgaggaaaggggcctgtcccgtgcagatatgcgatggggtgatgatgaggcatgcaggtttcctctcttcgacttcatgcaacccttgcccctgcacatgatggtcctgctgtacctgatcatgctgattggaacaggaggaattctattaggagccaagtaccgtgtgtgctgcgttatgcacctgctgccctactggtacatagtgcttctggacgagtgcagttggaacaatcactcctatctgtttggtctcctctctttcctccttctgctttgcgatgctaaccactactggtccatggacggtctgttcaatgccaaggttcgaaatacggatgttcccttgtggaactacaccctcctacgtacacaggtgtttctggtgtactttttggctgggctgaagaaactggacatggactggatcgctggttactccatgggccgtttgagtgatcattgggtcttttacccgtttacgttcctgatgacagaagaccaggtgagtgtgcttgtggtccacctgggtggacttgccattgacttgttcgtgggctacctgctcttctttgacaagacacgaccgatcggtgtcattatcagttcgtcattccacctgatgaatgcacagatgttcagcataggaatgtttccgtatgccatgttgggtttgacgcctgtgttcttctatgccaactggccgagggccctgtttcgccgcattccacgatccttgaggattcttacccctgatgatggagaggatgatacgctgccttcggagaagtgcttatacacaaaagaacaggccaaaccagaactggccagcacccctgagcatgaaaacactgcagtccgcaaacagttgacaccacccactcagcccacgttccggcatcatgctgccgctgccttcaccgttttcttcattctgtggcagatgtttttgcctttctctcattttatcacaaagggcaacaacagctggacccagggactctacggctactcctgggacatgatggttcacacccgcagcactcagcacaccaggatctccttcatcaacaaggacacaggagagcgagggttcctggacccgcaggcatggagcaagtcacatcgatgggcgcataacgctaagatgatgaagcagtacgccaggtgcatcgctcgccgactgaagaagcatgaaatcgacaatgtggaaatctattttgatgtctggatatctctgaatcatcgcttccagcaacggatcgtgaaccccaatgtggacattttaacagccgaatggagtgtctttaagtccactccatggatgatgcccttgctggtcgacttgtctaattggcgaagcaagttgaaagagattgaggacgacattttcaactcaaccgacctgtatgaaatagtctttctggctgactttcctggtttgtacctggagaactttgtccacggcagcgtcgggagtctcaacatctctgtactgcagggccaggtggtggtggaggtgcttccagaggaggacagtctagaagagccctacaacatcagcatcagtgatggccaagagtcattgattcccacaggggtgttccacaaggtgtacacagtgtctgaagtgccctcctgttacatgtacatctacatggtcacggaagagacagagttccttgagaaactcaaagagctggaacacgccctcaacggctccctggatgctccagttccagacaagtttgccgaagatcctaaacttgatcagtatatggaggtactcaaaacgaagaatgcaactccaccaccaacctctcaagaggagcaaagtttcatacagctgtttatgagttttctgaaaatgcattatatgtctatgtatcgtggactgcagctgataaaaggcgccatgtggtccatgtactccggggaatcttaccgagagttcttgaagaaactggagctacagaaaatgctggcggagaatgccaccctggtggcaaacgccacccaaggggtgaataacacccagacgatgaacaacaccttgaacaacaccaaggagaaagacaacacccaaagggttaacaaaccgcaggaaaagaaggccccccagaaggcagacagcccctaacagcatctctgcagaatgagggcgtcatgcctctgttctgcattgtaaatcttcaatgtcagactcgctgtcatgagtcaggatgccaagggttgattctaaatgaaaaaa

SEQ ID NO: 4 - protein, 811 aa, Conus textile
MQRPGKKVAADSEESNDISQQAENRDQLLPQEASPKACEEEDTEDEEEEEDKFYKLFGFSLSDLKSWDSFVRLLSRPADPAGLAYIRVTYGFLMMWDVFEERGLSRADMRWGDDEACRFPLFDFMQPLPLHMMVLLYLIMLIGTGGILLGAKYRVCCVMHLLPYWYIVLLDECSWNNHSYLFGLLSFLLLLCDANHYWSMDGLFNAKVRNTDVPLWNYTLLRTQVFLVYFLAGLKKLDMDWIAGYSMGRLSDHWVFYPFTFLMTEDQVSVLVVHLGGLAIDLFVGYLLFFDKTRPIGVIISSSFHLMNAQMFSIGMFPYAMLGLTPVFFYANWPRALFRRIPRSLRILTPDDGEDDTLPSEKCLYTKEQAKPELASTPEHENTAVRKQLTPPTQPTFRHHAAAAFTVFFILWQMFLPFSHFITKGNNSWTQGLYGYSWDMMVHTRSTQHTRISFINKDTGERGFLDPQAWSKSHRWAHNAKMMKQYARCIARRLKKHEIDNVEIYFDVWISLNHRFQQRIVNPNVDILTAEWSVFKSTPWMMPLLVDLSNWRSKLKEIEDDIFNSTDLYEIVFLADFPGLYLENFVHGSVGSLNISVLQGQVVVEVLPEEDSLEEPYNISISDGQESLIPTGVFHKVYTVSEVPSCYMYIYMVTEETEFLEKLKELEHALNGSLDAPVPDKFAEDPKLDQYMEVLKTKNATPPPTSQEEQSFIQLFMSFLKMHYMSMYRGLQLIKGAMWSMYSGESYREFLKKLELQKMLAENATLVANATQGVNNTQTMNNTLNNTKEKDNTQRVNKPQEKKAPQKADSP

SEQ ID NO: 5 - DNA, 612 bp, Conus omaria
gggagtctcaacatctctgtactgcagggccaggtggtggtggaggtgcttccagaggaggacagtctagaaaaaccctacaacatcagcatcaatgatggccacgagtcattgattcccacaggggtattccacaaggtgtacacagtgtctgaagtgccctcctgttacatgtacatctacatggtcacggaagagacggagttctttgagaacgtcaaagagctggaacacgccctcaacggctccctggatgctccagttccagacaagtttgccaaagatcctaaacttgatcaatatatggaggtactcaaagtgaagaatgcagctccaccaccggcccctcgagcggagagaagtttcatagagctgtttatgagttttctgaaaatgcattatatgtctatgtatcgtggactgcagctgataaaaggcgccgtgtggtccatgtactctggggaatcttaccgagagtacctgaaggaactggaattacaggcaatgctgggggagaatgccaccctggtggcaaacgccacccaaggggtgaataacacccagacgatgaacaacaccttattgaacaacaccaaaaaaaaaaaaaaaaaa

SEQ ID NO: 6 - protein, 204 aa, Conus omaria
GSLNISVLQGQVVVEVLPEEDSLEKPYNISINDGHESLIPTGVFHKVYTVSEVPSCYMYIYMVTEETEFFENVKELEHALNGSLDAPVPDKFAKDPKLDQYMEVLKVKNAAPPPAPRAERSFIELFMSFLKMHYMSMYRGLQLIKGAVWSMYSGESYREYLKELELQAMLGENATLVANATQGVNNTQTMNNTLLNNTKKKKKK

SEQ ID NO: 7 - DNA, 780 bp, Conus omaria
  CDS (1)..(666)
gggagtctcaacatctctgtactgcagggccaggtggtggtggaggtgcttccagaggaggacagtctagaaaaaccctacaacatcagcatcaatgatggccacgagtcattgattcccacaggggtattccacaaggtgtacacagtgtctgaagtgccctcctgttacatgtacatctacatggtcacggaagagacggagttctttgagaacgtcaaagagctggaacacgccctcaacggctccctggatgctccagttccagacaagtttgccaaagatcctaaacttgatcaatatatggaggtactcaaagtgaagaatgcagctccaccaccggcccctcgagcggagagaagtttcatagagctgtttatgagttttctgaaaatgcattatatgtctatgtatcgtggactgcagctgataaaaggcgccgtgtggtccatgtactctggggaatcttaccgagagtacctgaaggaactggaattacaggcaatgctgggggagaatgccaccctggtggcaaacgccacccaaggggtgaataacacccagacgatgaacaacaccttattgaacaacaccaaggagaaaaacaacacccaaagggttaacaagccgcaggaaaagaaggccccccagaaggcagacagcccctaacagcatctctgcagaatgagggcatcatgcctctgttctgcattttaaatcttcgatgtcagacacgctgtcatgagtcaggatgccaagggttgattctaaatgaaaaaa

SEQ ID NO: 8 - protein, 222 aa, Conus omaria
GSLNISVLQGQVVVEVLPEEDSLEKPYNISINDGHESLIPTGVFHKVYTVSEVPSCYMYIYMVTEETEFFENVKELEHALNGSLDAPVPDKFAKDPKLDQYMEVLKVKNAAPPPAPRAERSFIELFMSFLKMHYMSMYRGLQLIKGAVWSMYSGESYREYLKELELQAMLGENATLVANATQGVNNTQTMNNTLLNNTKEKNNTQRVNKPQEKKAPQKADSP

SEQ ID NO: 9 - DNA, 609 bp, Conus episcopatus
gggagtctcaacatctctgtactgcagggccaggtggtggtggaggtgcttccagaggaggacagtctagaacagccctacaacatcagcatcagtgatggccacgagtcattgattcccacaggggtgttccacaaggtgtacacagtgtctgaagtgccctcctgttacatgtacatctacatggtcacggaagagacggagttctttgagaacctcaaagagctggaacacgccctcaacggctccctggatgctccagtaccagacaagtttgccaaagatcctaaacttgatcaatatatggaggtactcaaggtgaagaatgcagctccaccaccggcccctccagcggacagaagtttcatacagctgtttatgagttttctgaaaatgcattatatgtctatgtatcgtggactgcagctgataaaaggcgccgtgtggtccatgtactctggggaatcttaccgagagtacctgaaggaactggagctacaggcaatgctgggggagaatgtcaccctggtggcaaatgccacccaaggggtgaataaaacccagatgatgaacaacaccttgaacaacaccaaaaaaaaaaaaaaaaaa

SEQ ID NO: 10 - protein, 203 aa, Conus episcopatus
GSLNISVLQGQVVVEVLPEEDSLEQPYNISISDGHESLIPTGVFHKVYTVSEVPSCYMYIYMVTEETEFFENLKELEHALNGSLDAPVPDKFAKDPKLDQYMEVLKVKNAAPPPAPPADRSFIQLFMSFLKMHYMSMYRGLQLIKGAVWSMYSGESYREYLKELELQAMLGENVTLVANATQGVNKTQMMNNTLNNTKKKKKK

SEQ ID NO: 11 - DNA, 777 bp, Conus episcopatus
  CDS (1)..(663)
gggagtctcaacatctctgtactgcagggccaggtggtggtggaggtgcttccagaggaggacagtctagaacagccctacaacatcagcatcagtgatggccacgagtcattgattcccacaggggtgttccacaaggtgtacacagtgtctgaagtgccctcctgttacatgtacatctacatggtcacggaagagacggagttctttgagaacctcaaagagctggaacacgccctcaacggctccctggatgctccagtaccagacaagtttgccaaagatcctaaacttgatcaatatatggaggtactcaaagtgaagaatgcagctccaccaccggcccctccagcggacagaagtttcatacagctgtttatgagttttctgaaaatgcattatatgtctatgtatcgtggactgcagctgataaaaggcgccgtgtggtccatgtactctggggaatcttaccgagagtacctgaaggaactggagctacaggcaatgctgggggagaatgtcaccctggtggcaaatgccacccaaggggtgaataaaacccagatgatgaacaacaccttgaacaacaccaaggagaaaaacaacacccaaagggttaacaagccgcaggaaaagaaggccccccagaaggcagacagcccctaacagcatctctgcagaatgagggcatcatgcctctgttctgcattttaaatcttcaatgtcagacacgctgtcatgagtcaggatgccaagggttgattctaaatgaaaaaa

SEQ ID NO: 12 - protein, 221 aa, Conus episcopatus
GSLNISVLQGQVVVEVLPEEDSLEQPYNISISDGHESLIPTGVFHKVYTVSEVPSCYMYIYMVTEETEFFENLKELEHALNGSLDAPVPDKFAKDPKLDQYMEVLKVKNAAPPPAPPADRSFIQLFMSFLKMHYMSMYRGLQLIKGAVWSMYSGESYREYLKELELQAMLGENVTLVANATQGVNKTQMMNNTLNNTKEKNNTQRVNKPQEKKAPQKADSP